SEQUENCE LISTING (6 sequences)

SEQ ID NO: 1 - protein, 9 aa, Homo sapiens
EYQAAYGKE

SEQ ID NO: 2 - protein, 15 aa, Homo sapiens
  MISC_FEATURE (1)..(15): peptide scan peptide spot No. 17
NAQRQLIVKEYQAAY

SEQ ID NO: 3 - protein, 15 aa, Homo sapiens
  MISC_FEATURE (1)..(15): peptide scan peptide spot No. 18
RQLIVKEYQAAYGKE

SEQ ID NO: 4 - protein, 15 aa, Homo sapiens
  MISC_FEATURE (1)..(15): peptide scan peptide spot No. 19
IVKEYQAAYGKELKD

SEQ ID NO: 5 - protein, 15 aa, Homo sapiens
  MISC_FEATURE (1)..(15): peptide scan paptide spot No. 20
EYQAAYGKELKDDLK

SEQ ID NO: 6 - protein, 15 aa, Homo sapiens
  MISC_FEATURE (1)..(15): peptide scan peptide spot No. 21
AAYGKELKDDLKGDL

(19) United States
(12) Patent Application Publication
(10) Pub. No.: US 2011/0177536 A1
(43) Pub. Date: Jul. 21, 2011

(54) NOVEL ANTIBODIES RECOGNIZING NATIVE ANNEXIN A3

(75) Inventors: Slobodan Poznanovic, Mainz (DE); Gerhard Schwall, Mainz (DE)

(73) Assignee: PROTEOSYS AG, Mainz (DE)

(21) Appl. No.: 13/121,011

(22) PCT Filed: Sep. 25, 2009
(86) PCT No.: PCT/EP2009/062470

§ 371 Date: Mar. 25, 2011

Related U.S. Application Data
(60) Provisional application No. 61/100,367, filed on Sep. 26, 2008.

(51) Int. Cl. G01N 33/53; C07K 16/18

(52) U.S. Cl. 435/7.92; 530/387.9; 530/387.3

(57) ABSTRACT

The present invention refers to novel antibodies recognizing native annexin A3. These antibodies are suitable for diagnostic and therapeutic applications.

The present invention refers to novel antibodies recognizing native annexin A3. These antibodies are suitable for diagnostic and therapeutic applications.

In a previous studies it was shown that ANXA3 is significantly associated a with the progression and the various stages of prostatic disease and prostate cancer. Still, little is known about the role and biology of ANXA3 in the human prostate. So far it has been shown that ANXA3 is part of so-called exosomes, small vesicles released form a variety of epithelia. There are indications that this phenomenon, which is the basis of the diagnostic performance of ANXA3, is due to a complex regulation of autoimmunity in prostate cancer.

Protein biomarkers for therapy or diagnostics of prostate cancer and other epithelial cancers of urogenital tract are described in US 2005 130 876, WO 03 086 461, WO 2005 078 124, EP 05 011 042.8 and EP 05 026 092.6. The content of these documents is herein incorporated by reference.

U.S. 60/812,089 and U.S. 60/859,489 disclose the diagnosis of cancer, wherein a sample is analyzed for the presence and/or amount of annexin A3 with highly specific monoclonal antibodies. The content of these documents is herein incorporated by reference.

Although ANXA3 has been known as marker for prostate cancer and specific antibodies for methods concerning differential diagnosis between benign, premalignant and malignant conditions in tissue sections are available, the measurement of ANXA3 in urine and exprimate urine by these antibodies was met with a surprising difficulty.

The conformation of “native” ANXA3 in urine is such that currently available antibodies, which are highly specific in Western blots or tissue staining, only detect a fraction of the protein. This is probably due to folding of ANXA3 in exosomes/prostasomes which results in non-accessibility of epitopes which are important for specific recognition of the protein. These epitopes have been explored and described in detail in a previous patent application.

The aim of the present study was to investigate the quantitatively the abundance of native ANXA3 in a body fluid such as urine and exprimate urine.

According to the present invention it was found that an efficient detection of ANXA3 in urine using the previously described anti ANXA3-antibodies which had prognostic relevance was only possible by employing denaturing conditions and Western blot techniques. None of these previously described antibodies worked well in ELISA-based tests, where exprimate urine was tested under “native” conditions.

Surprisingly, we found out, that next to the epitopes making up the complete specific “antibody-reactive surface” of ANXA3 and described previously, antibodies recognizing “native” ANXA3 in urine, exprimate urine and other body fluids (like plasma or serum) have an additional epitope for specific recognition native ANXA3.

In a first aspect, the present invention refers to antibodies recognizing native annexin A3, particularly native human annexin A3. These antibodies are capable of specific binding to ANXA3 under non-denaturing conditions, i.e. in body fluids, such as urine and exprimate urine.

In a first preferred embodiment, the invention refers to an antibody recognizing native annexin A3, which is directed against an epitope in the amino acid sequence 59-67 of human annexin A3:

| | |
|---|---|
| E Y Q A A Y G K E, | (Seq. ID NO: 1) |

or an antigen-binding fragment of said antibody.

Specific examples of such antibodies are selected from:
(i) the antibody
   TGC42
   TGC43 or
   TGC49
(ii) an antibody having the same antigen binding site as the antibody from (i) and
(iii) an antigen-binding fragment of said antibody.

A further preferred embodiment refers to an antibody recognizing native annexin A3, which is directed against a conformational epitope in the amino acid sequence 1-106 of human annexin A3, or an antigen-binding fragment of said antibody.

For example, this antibody may be directed against a conformational epitope in the amino acid sequence 1-34 of human annexin A3, or an antigen-binding fragment of said antibody. Alternatively, this antibody may be directed against a conformational epitope in the amino acid sequence 35-106 of human annexin A3, or an antigen-binding fragment of said antibody.

Specific examples for such antibodies are selected from
(i) the antibody
   TGC44 or
   TGC48,
(ii) the antibody having the same antigen-binding site as the antibody from (i) and
(iii) an antigen-binding fragment of said antibody.

In a still further embodiment the antibody of the present invention is selected from
(i) the antibody TGC42, TGC43, TGC44, TGC45, TGC46, TGC47, TGC48 or TGC49,
(ii) an antibody having the same binding site as the antibody from (i) and
(iii) an antibody recognizing the same epitope on native human annexin A3 as the antibody from (i) or (ii), and
(iv) an antigen-binding fragment of said antibody.

Hybridoma cell lines producing antibodies TGC42 (DSM ACC 2972), TGC43 (DSM ACC 2970), TGC44 (DSM ACC 2976), TGC45 (DSM ACC 2974), TGC46 (DSM ACC 2975), TGC47 (DSM ACC 2977), TGC48 (DSM ACC 2971) and TGC49 (DSM ACC 2973) were deposited under the conditions of the Budapest Treaty at DSMZ (Deutsche Sammlung von Mikroorganismen und Zellkulturen GmbH), Inhoffenstr. 7 B, 38124 Braunschweig, on 17 Sep. 2008.

The antibody of the invention may be a monoclonal, chimeric, humanized, human or recombinant antibody, e.g. a single chain antibody or an antigen-binding fragment of such an antibody, e.g. a proteolytic fragment or a recombinant single chain antibody fragment. The antibody is useful in medicine, particularly in human medicine. More particularly, the antibody may be used in diagnostic or therapeutic applications. Most preferably, the antibody is used for the diagnosis of cancer, such as prostate cancer. Advantageously, the antibody of the present invention is for use in a diagnostic assay which is performed under native conditions, e.g. a capture assay such as an ELISA wherein the presence and/or amount of native annexin A3 is determined.

For therapeutic and diagnostic applications, the antibody of the present invention may be conjugated to effector groups or labelling groups as known in the art. Effector groups may e.g. be selected from cytotoxic groups or compounds, e.g. chemotherapeutic agents or radionuclides. Labelling groups may be selected from any known labelling groups such as fluorescent groups, luminescent groups, enzyme labels, radioactive labels etc. The coupling of effector or labelling groups to antibodies may be carried out according to known techniques in the art.

In a further aspect, the present invention refers to a method for the diagnosis of cancer, preferably prostate cancer, wherein a body fluid sample is analyzed for the presence and/or amount of native annexin A3.

Preferably, the diagnostic method of the present invention involves a detection of native annexin A3, particularly native human annexin A3, by reaction with an antibody as described above. The sample is preferably a body fluid, such as urine or exprimate urine.

The cancer which may be diagnosed according to the present invention is preferably a cancer of the urogenital and/or gastro-intestinal tract, such as cancer of prostate, bladder, kidney, urethra, ovaria, uterus or colon. Particularly, the cancer is an epithelial cancer. In an especially preferred embodiment, the cancer is prostate cancer.

In a preferred embodiment, the invention comprises a differential diagnosis of a disease stage and/or a prognostic evaluation. For example, the present invention allows a differential diagnosis of disease stages selected from:
(i) a benign condition, particularly a benign prostatic condition such as benign prostatic hyperplasia (BPH), fibrosis and chronic prostatitis,
(ii) a premalignant condition such as prostatic intraepithelial neoplasia of various stages (PIN-1-3) including high-grade prostatic intraepithelial neoplasia (HGPIN), and/or
(iii) a malignant condition such as prostate cancer, particularly advanced prostate cancer in a progressed state indicated by Gleason rating.

More preferably, the invention allows a differential diagnosis between a benign or premalignant condition on the one hand and a malignant condition on the other hand.

In a preferred embodiment of the invention, the presence, amount and/or distribution of native ANXA3 is determined in a sample, which may be a body fluid or a tissue sample. It should be noted that presence, amount and intracellular localization may be determined on a single tissue sample or on different samples, e.g. a body fluid sample and a tissue sample from the same subject.

A high amount of native ANXA3 in a sample, e.g. corresponding to a strong staining of a tissue sample, is primarily indicative for a benign condition. A moderate/low amount of native ANXA3, e.g. corresponding to weak/moderate staining of a tissue sample, is indicative for a premalignant condition or to a early/early-intermediate stage malignant condition. The absence of native ANXA3 in a sample is indicative for a malignant condition, primarily for a malignant condition in a progressed state, e.g. in an intermediate or late stage.

The presence of native ANXA3 in vesicles, e.g. exosomes and/or prostatosomes, is primarily indicative for a benign or premalignant condition. These vesicles can be obtained from urine or other body fluids by differential centrifugation, so the present invention relates to detection of native ANXA3 in any fraction of said body fluids.

Further, the method of the invention may encompass the determination of auto-antibodies against annexin A3.

The presence of an autoimmune response against ANXA3, e.g. the presence of auto-antibodies against ANXA3, is primarily indicative for a malignant condition, particularly for a malignant condition in a progressed state.

The present invention encompasses determination of polypeptides in a sample. Preferably, this determination comprises immunological methods, wherein the presence, amount and/or localization of a component in a sample is determined using immunological test reagents.

The reagent for determining native ANXA3 in a sample is preferably an antibody which is specific for native ANXA3, e.g. a polyclonal or a monoclonal antibody as described above.

The reagent for determining ANXA3 auto-antibodies in a sample is preferably an ANXA3 polypeptide or a fragment thereof comprising specific epitopes important for recognition. More preferably, the ANXA3 polypeptide is a recombinant ANXA3 polypeptide, refolded to a native state, or native ANXA3 isolated from human or other mammalian sources.

The method of the present invention may be carried out in any test format suitable for immunological determinations, including test formats suitable for automated devices. In some test formats it may be preferred to use a reagent which carries a labelling group, e.g. a visual marker, such as a latex or gold bead, a fluorescence marker group, an enzymatic marker group etc.

Conjugates of reagents and labelling groups may be produced according to standard methods, e.g. by covalent coupling to reactive amino acid side groups of the reagent such as carboxy, amino and/or thiol groups with labelling groups, e.g. via bifunctional spacer molecules.

The sample is preferably obtained from a human subject. In some embodiments, the method is a non-invasive diagnostic procedure, wherein the sample may be e.g. a urine sample, particularly an urine sample, an exprimate urine sample or a faeces sample. If desired, the sample may be subjected to pretreatment procedures, e.g. gel filtration. In further embodiments, the method may be a histochemical procedure wherein the sample may be a tissue sample, particularly a biopsy, e.g. a punch biopsy. In a histochemical procedure, a selective determination of intracellular or extracellular ANXA3 may be carried out by determining the localisation of ANXA3 within the sample.

In a preferred embodiment, the invention comprises a semi-quantitative or quantitative determination of native ANXA3. This semi-quantitative or quantitative determination may involve evaluation of test results based on predetermined cut-off values and correlating the results of the evaluation with a disease stage. Cut-off values may be determined by determining native ANXA3 in samples from healthy persons and/or persons with a predetermined disease stage according to known methods. Further, the amount of native ANXA3 in a sample may also be determined by evaluation of stained tissue samples and correlating the results of the evaluation with a disease stage.

The sample may be subjected to a fractionation procedure which allows separate determination of native ANXA3, e.g. in exosomes. For example, the sample may be centrifuged in order, to obtain a cell pellet and a supernatant whereby intracellular annexin A3 is determined in the cell pellet and extracellular annexin A3 is determined in the supernatant. In an especially preferred embodiment the method comprises a selective determination of extracellular ANXA3. In a further especially preferred embodiment, the method comprises a selective determination of intracellular ANXA3.

The method of the present invention may additionally comprise the determination of further cancer markers, e.g. cancer markers. The determination of further markers may be carried out in the same sample where native ANXA3 is determined or in different samples, e.g. blood, serum and/or plasma samples. Especially preferred is the determination of blood, serum or plasma markers, in particular of at least one member of the kallikrein protease family, such as prostate specific antigen (PSA) and/or at least one epithelial cell marker, particularly prostate specific membrane antigen (PSMA).

The present invention may be a screening procedure, wherein an individual or a group of individuals are tested for cancer, particularly prostate cancer. On the other hand, the method may also comprise a prognostic evaluation or therapeutic follow-up testing, wherein an individual who already has been diagnosed positive for cancer, particularly prostate cancer or a precursor stage thereof, is subjected to a prognostic evaluation and/or a therapy control monitoring.

In an especially preferred embodiment, the invention relates to a prognostic evaluation of the disease progression, which is a valuable tool in any diagnostic assessment, particularly for therapy control. The prognostic evaluation may be based on determination of native ANXA3 alone or in combination with other markers such as PSA, PCA3 (Loeb S. Does PCA3 Help Identify Clinically Significant Prostate Cancer? Eur Urol. 2008 Jul. 16.), PCADM-1 (Ohkia A, Hu Y, Wang M, Garcia F U, Stearns M E. Clin Cancer Res. 2004 Apr. 1; 10(7):2452-8. Links Evidence for prostate cancer-associated diagnostic marker-1: immunohistochemistry and in situ hybridization studies.), EPCA-2 (Katz M D, Kibel A S. Words of wisdom. Re: EPCA-2: a highly specific serum marker for prostate cancer. Eur Urol. 2008 January; 53(1):210), (Diamandis EP. POINT: EPCA-2: a promising new serum biomarker for prostatic carcinoma? Clin Biochem. 2007 December; 40(18)1437-9. Epub 2007 Sep. 19.), (Leman E S, Cannon G W, Trock B J, Sokoll L J, Chan D W, Mangold L, Partin A W, Getzenberg R H. EPCA-2: a highly specific serum marker for prostate cancer. Urology. 2007 April; 69(4):714-20), which are correlated positively to prostate cancer. For example, by histological evaluation and/or by measuring levels of PSA or further cancer markers, patients may be classified in a low risk group (e.g. PSA level ≦10 ng/ml), an intermediate risk group (e.g. PSA level >10 and <20 ng/ml) and a high risk group (e.g. PSA level ≧20 ng/ml). Determination of ANXA3 in these patient groups may lead to further valuable information, particularly in patients having been classified as being in an intermediate risk group. If these intermediate risk patients are ANXA3 positive, the percentage of PSA-free survival is significantly higher than in patients having been determined as being negative for ANXA3. Thus, the invention allows a further risk stratification for individual patient groups. Preferably, patients in a group having originally been classified as being in an intermediate risk group, may be reclassified based on the results of the ANXA3 determination. Patients who are ANXA3 positive may be reclassified as being in a low risk group and patients who are ANXA3-negative (and optionally have an autoimmune response against ANXA3) are classified as being in a high risk group.

Further, the present invention shall be explained in more detail by the following Figures and Examples.

### EXAMPLES

Mice were immunized with recombinant human annexin A3. Thereby, monoclonal antibodies were generated which are capable of recognizing native annexin A3 in a capture ELISA combined with the polyclonal antiserum Petros (Wozny W, Schroer K, Schwall G P, PoznanovićS, Stegmann W, Dietz K, Rogatsch H, Schaefer G, Huebl H, Klocker H, Schrattenholz A, Cahill M A. Differential radioactive quantification of protein abundance ratios between benign and malignant prostate tissues: cancer association of annexin A3. Proteomics. 2007 January; 7(2):313-22).

The resulting antibodies were designated as follows:
TGC43=DSM ACC2970
TGC48=DSM ACC2971
TGC42=DSM ACC2972
TGC49=DSM ACC2973
TGC45=DSM ACC2974
TGC46=DSM ACC2975
TGC44=DSM ACC2976
TGC47=DSM ACC2977

### 1. Localisation of Binding Sites in Western Blot.

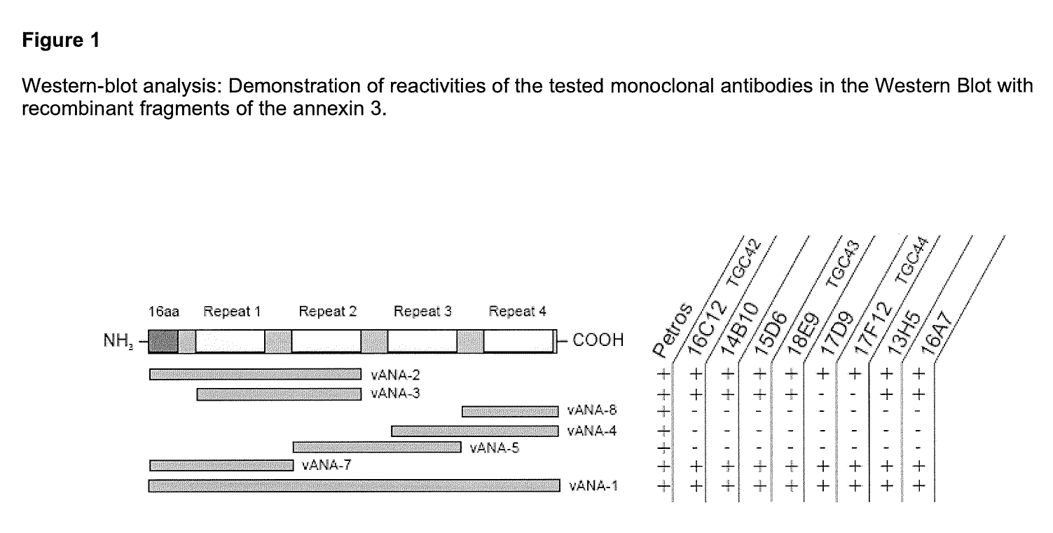

In a first set of experiments, cell culture supernatants of hybridoma cells 16C12 (TGC42), 14B10 (TGC49), 15D6 (TGC46), 18E9 (TGC43), 17D9 (TGC48), 17F12 (TGC44), 13H5 (TGC47) and 16A7 (TGC45) were tested in Western Blot with the recombinant annexin 3 fragments vANA1, vANA4, vANA5, vANA7 and vANA8. These fragments of annexin 3 represent the complete annexin 3 in overlapping fragments (cf. FIG. 1). All antibody supernatants recognize fragment vANA1 and, thus, the full-length annexin A3 protein. Moreover, all tested antibodies detect fragment vANA7 representing the N-terminal region of annexin 3 (cf. FIG. 1). The binding sites of the tested antibodies, thus, are within the N-terminal 106aa of annexin 3.

In order to further localise the binding sites of the monoclonal antibodies, the cell culture supernatants in a second set of experiments in the Western Blot were tested as to their reactivity to fragments vANA2 and vANA3 of annexin 3. vANA3 corresponds to fragment vANA2 except to the N-terminal region 34aa. Two of the monoclonal antibodies—17D9 (TGC48) and 17F12 (TGC44)—only recognize fragment vANA2, the remaining antibodies recognize both annexin 3 fragments (FIG. 1). These experiments showed that there are at least two different reactivities in the generated antibodies. Whereas in monoclonal antibodies 17D9 (TGC48) and 17F12 (TGC44) the reactivity is associated with N-terminal 34aa, the binding site of the remaining antibodies is between the amino acids 35 and 106 of the annexin 3 sequence.

2. Epitope Mapping of Selected Antibodies with Peptide Scan

Epitopes of monoclonal antibodies 16C12 (TGC42), 18E9 (TGC43), 17F12 (TGC44), 14B10 (TGC49), 17D9 (TGC48) as well as of various antibody supernatants were determined by peptide scan.

### 2.1 Epitope Mapping of Antibodies 16C12 (TGC42), 18E9 (TGC43) and 14B10 (TGC49)

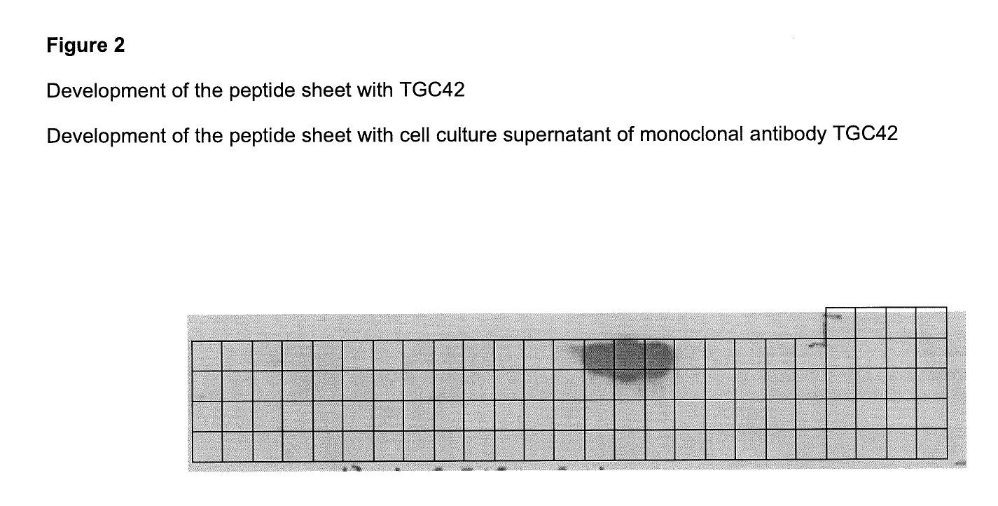

Cell culture supernatants of monoclonal antibodies 16C12 (TGC42), 18E9 (TGC43) and 14B10 (TGC49) were used in undiluted form for characterizing their epitopes. These three antibodies showed identical results in the peptide scan. A representative result of the development of the peptide sheet is shown in FIG. 2. The monoclonal antibodies 16C12 (TGC42), 18E9 (TGC43) and 14B10 (TGC49) recognize peptide spots 18-20 of the peptide sheet.

Analysis of the development of the peptide sheet

| | | |
|---|---|---|
| 17 | NAQRQLIVKEYQAAY | − |
| 18 | RQLIVKEYQAAYGKE | ++ |
| 19 | IVKEYQAAYGKELKD | +++ |
| 20 | EYQAAYGKELKDDLK | ++ |
| 21 | AAYGKELKDDLKSDL | − |

The epitope of the monoclonal antibody 16C12 (TGC42), 18E9 (TGC43) and 14B10 (TGC49) is within the amino acid sequence EYQAAYGKE (positions 59-67 of annexin A3).

### 2.2 Epitope Mapping of Antibodies 17D9 (TGC48) and 17F12 (TGC44)

Cell culture supernatants of monoclonal antibodies 17D9 (TGC48) and 17F12 (TGC44) were used in undiluted form for characterizing their epitopes. Both antibody supernatants did not show spots in the peptide scan. Apparently, both antibodies did not realize a linear epitope, but a conformational epitope of annexin A3.

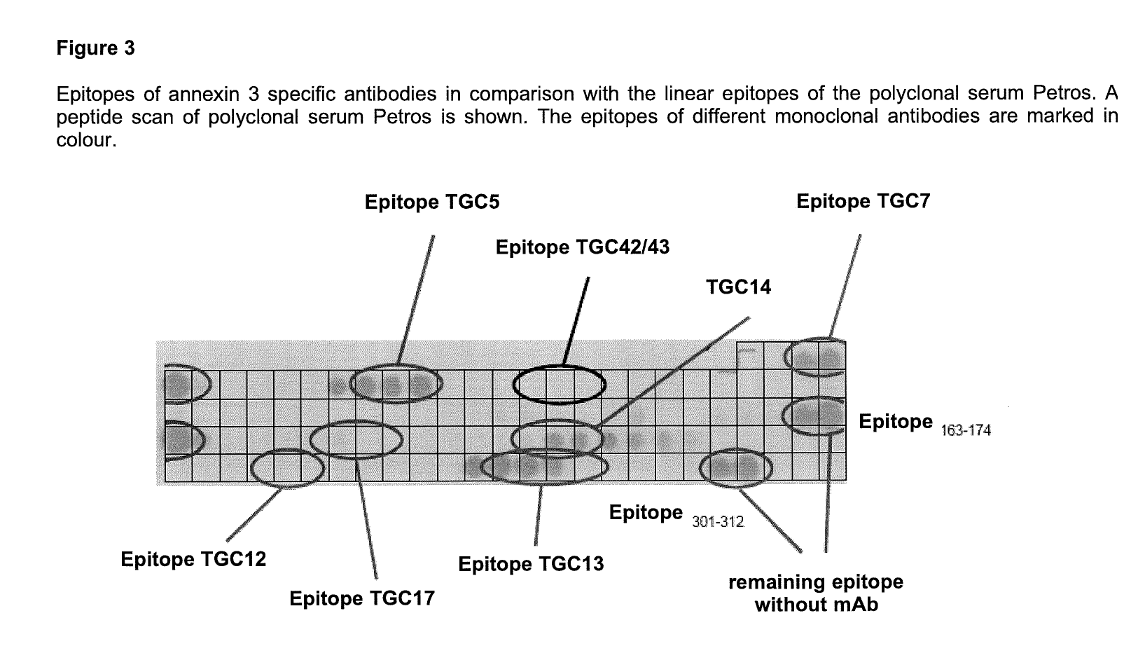

Based on these results, the conformational epitope recognized by the antibodies 17D9 (TGC48) and/or 17F12 (TGC44) may be located within the amino acids 1-106, 1-34 and/or 35-106 of human annexin A3. FIG. 3 shows epitopes recognized by ANXA3 specific antibodies in comparison to linear epitopes of the polyclonal anti-serum Petros. The figure shows a peptide scan of the polyclonal serum PETROS. The epitopes of different monoclonal antibodies are indicated.

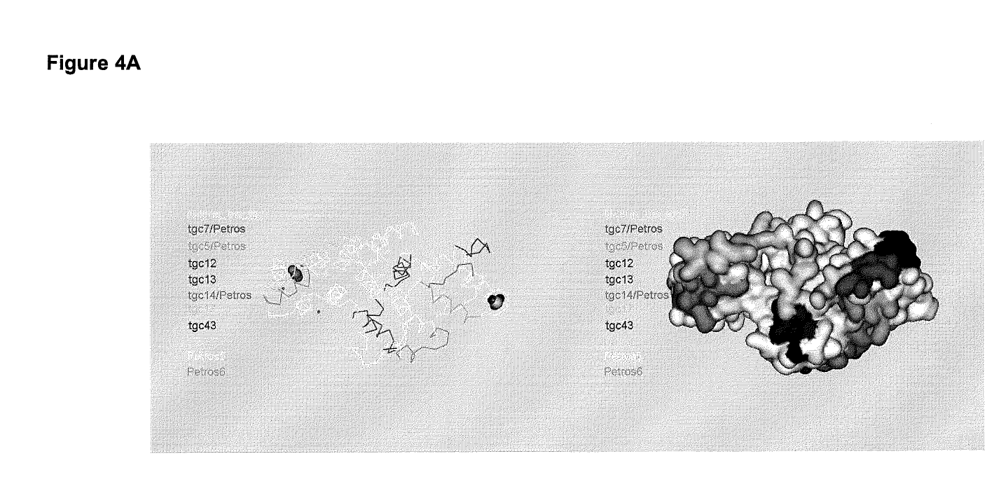

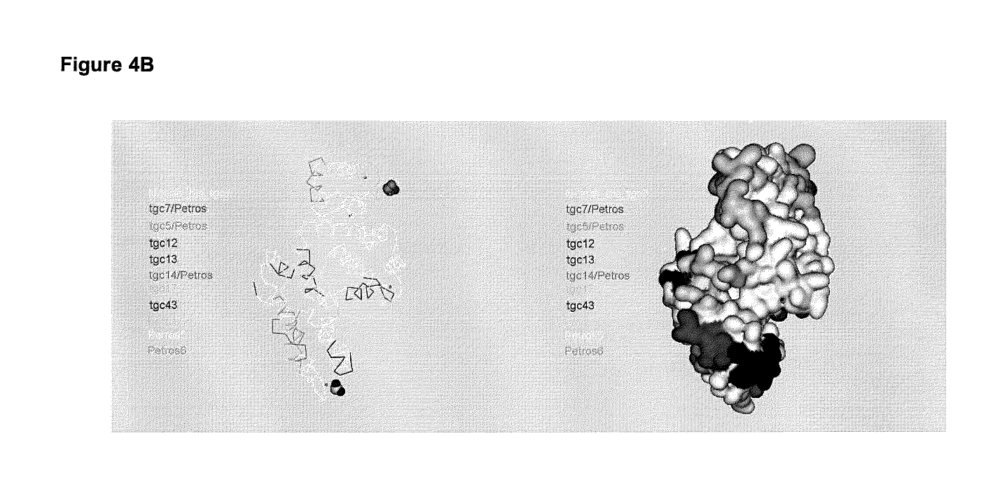

FIG. 4 is a depiction of the ANXA3 spatial structure indicating epitopes recognized by antibodies recognizing denatured ANXA3 and antibodies recognizing native ANXA3 (present invention).

1. An antibody recognizing native annexin A3, which is directed against an epitope in the amino acid sequence 59-67 of human annexin A3:

| | |
|---|---|
| E Y Q A A Y G K E, | (Seq. ID NO: 1) |

or an antigen-binding fragment of said antibody.

2. The antibody of claim 1 which is selected from:
(i) antibody
TGC42
TGC43 or
TGC49
(ii) an antibody having the same antigen binding site as the antibody from (i) or
(iii) an antigen-binding fragment of said antibody.

3. An antibody recognizing native annexin A3, which is directed against a conformational epitope in the amino acid sequence 1-106 of human annexin A3, or an antigen-binding fragment of said antibody.

4. The antibody of claim 3 which is directed against a conformational epitope in the amino acid sequence 1-34 of human annexin A3, or an antigen-binding fragment of said antibody.

5. The antibody of claim 3 which is directed against a conformational epitope in the amino acid sequence 35-106 of human annexin A3, or an antigen-binding fragment of said antibody.

6. The antibody of claim 3 which is selected from
(i) the antibody
TGC44 or
TGC48,
(ii) an antibody having the same antigen-binding sites as the antibody from (i) or
(iii) an antigen-binding fragment of said antibody.

7. An antibody recognizing native annexin A3 selected from
(i) the antibody TGC42, TGC43, TGC44, TGC45, TGC46, TGC47, TGC48 or TGC49,
(ii) an antibody having the same binding site as the antibody from (i)
(iii) an antibody recognizing the same epitope on native human annexin A3 as the antibody from (i) or (ii), or
(iv) an antigen-binding fragment of said antibody.

8. The antibody of claim 1 which is a monoclonal, dimeric, humanized, human or recombinant antibody or a fragment thereof.

9. The antibody of claim 1 for use in medicine, particularly in human medicine.

10. The antibody of claim 9 for use in diagnostics or therapy.

11. The antibody of claim 9 for the diagnosis of cancer, particularly prostate cancer.

12. The antibody of claim 9 for use in a diagnostic assay which is performed under native conditions.

13. The antibody of claim 12 for use in an ELISA.

14. A method for the diagnosis of cancer comprising analyzing a sample to detect the presence and/or amount of native annexin A3.

15. The method of claim 14, wherein the native annexin A3 is detected by reaction with an antibody recognizing native annexin A3, which is directed against an epitope in the amino acid sequence 59-67 of human annexin A3:

| | |
|---|---|
| E Y Q A A Y G K E, | (Seq. ID NO: 1) |

or an antigen-binding fragment of said antibody.

16. The method of claim 14 wherein the sample is a body fluid, particularly urine or exprimate urine.

17. The method of claim 15, wherein the antibody is selected from
(i) antibody
TGC42
TGC43 or
TGC49
(ii) an antibody having the same antigen binding site as the antibody from (i) or
(iii) an antigen-binding fragment of said antibody.

18. The method of claim 15, wherein the antibody is a monoclonal, dimeric, humanized, human or recombinant antibody or a fragment thereof.

19. The method of claim 14, wherein the native annexin A3 is detected by reaction with an antibody recognizing native annexin A3, which is directed against a conformational epitope in the amino acid sequence 1-106 of human annexin A3, or is an antigen-binding fragment of said antibody.

20. The method of claim 19, wherein said antibody is directed against a conformational epitope in the amino acid sequence 1-34 of human annexin A3, or is an antigen-binding fragment of said antibody.

21. The method of claim 19, wherein said antibody is directed against a conformational epitope in amino acid sequence 35-106 of human annexin A3, or is an antigen-binding fragment of said antibody.

22. The method of claim 19, wherein said antibody is selected from
(i) antibody
TGC44 or
TGC48,
(ii) an antibody having the same antigen-binding sites as the antibody from (i) or
(iii) an antigen-binding fragment of said antibody.

23. The method of claim 14, wherein the native annexin A3 is detected by reaction with an antibody recognizing native annexin A3 selected from
(i) antibody TGC42, TGC43, TGC44, TGC45, TGC46, TGC47, TGC48 or TGC49;
(ii) an antibody having the same binding site as the antibody from (i);
(iii) an antibody recognizing the same epitope on native human annexin A3 as the antibody from (i) or (ii); or
(iv) an antigen-binding fragment of said antibody.
